(19) United States
(12) Patent Application Publication
(10) Pub. No.: US 2006/0252725 A1
(43) Pub. Date: Nov. 9, 2006

(54) Use of prebiotics for preventing or treating oxidation stress

(76) Inventors: Yves Rayssiguier, Romagnat (FR); Jéromes Busserolles, Le Vernet (FR); André Mazur, Coumon (FR); Elyett Guex, Saint Genes Champanelle (FR)

Correspondence Address: NIXON & VANDERHYE, PC, 901 NORTH GLEBE ROAD, 11TH FLOOR, ARLINGTON, VA, 22203, US

(21) Appl. No.: 10/539,632

(22) PCT Filed: Dec. 17, 2003
(86) PCT No.: PCT/FR03/03770

§ 371 Date: Nov. 9, 2005

(30) Foreign Application Priority Data
Dec. 18, 2002 (FR) 02/16136

(51) Int. Cl. A61K 31/715

(52) U.S. Cl. 514/54; 514/61

(57) ABSTRACT

The invention relates to the use of prebiotics for the preparation of food preparations, functional foods, or pharmaceutical compositions for preventing or treating oxidative stress in particular linked to the consumption of fructose. The invention also relates to a food preparation comprising simple carbohydrates, in particular fructose, in combination with prebiotics.

A subject of the present invention is the use of prebiotics for the preparation of food preparations, functional foods, or pharmaceutical compositions intended to prevent or treat oxidative stress.

Oxidative stress is the result of an imbalance in the organism in favor of pro-oxidant species relative to anti-oxidant species.

Pro-oxidant species are generally free radicals and in particular oxygenated free radicals. The presence of an unpaired electron makes these compounds extremely reactive vis-à-vis the biological macromolecules of the organism; lipids, carbohydrates, proteins and nucleic acids are thus preferred targets of these species. The oxidative degradation of these macromolecules leads to many cellular malfunctions.

The origin of these free radicals is essentially found in the metabolism of oxygen. Most of the oxidative stress originates from the energy metabolism. The final stage of the oxidation of food, namely the mitochondrial respiratory chain, is thus the origin of the formation of oxygenated free radicals. Moreover, during inflammatory reaction, the stimulation of the phagocytes is also accompanied by the formation of free radicals.

The anti-oxidant defenses of the organism use protein systems, such as superoxide dismutase, but also anti-oxidant compounds provided by food, such as vitamins C and E, or other nutrients, such as carotenoids, polyphenols or flavonoids.

Dietary imbalances can be the origin of oxidative stress. The inventors have in particular previously shown that food which is too rich in sugars, and in particular in saccharose (Busserolles et al., 2002a) and fructose (Busserolles et al., 2002b), could cause significant oxidative stress. These pro-oxidant effects are even more significant the lower the content of anti-oxidants in the food is.

Fructose is a monosaccharide, the consumption of which has greatly increased, either as it is or in the form of saccharose. Because of their low production costs, syrups that are rich in fructose produced from corn are preferably used in sugary drinks. While fructose is naturally present in honey and in fruits where it is associated with many protective micronutrients, the consequences of an unrestricted increase of this carbohydrate in purified form on health are questionable. In fact, fructose has many properties which distinguish it from other sugars and the high intake of this carbohydrate could be responsible for undesirable metabolic effects.

The modalities for combating oxidative stress generally include the use of anti-radical nutrients having a direct effect on free radicals: carotenes, ascorbic acid (vitamin C), tocopherols (vitamin E), polyphenols (U.S. Pat. No. 6,207,702).

The present invention results from the inventors demonstrating that prebiotics and more particularly fructooligosaccharides (FOS) can combat the oxidative stress resulting from an excess of fructose in food.

Prebiotics are non-digestible complex carbohydrates broken down by the microorganisms of the intestinal flora and whose breakdown has beneficial health effects for the host. These microorganisms are generally bacteria, and in particular bifidobacteria, which are essentially found in the colon. The beneficial effects provided by said microorganisms can be due to the selective stimulation of the growth of certain species of microorganisms, in particular bifidobacteria, and/or to the release of metabolites originating from the conversion of the prebiotics by the microorganisms.

At present, the only clearly defined prebiotics are sugar polymers with a degree of polymerization comprised between 2 and 12, classified as complex carbohydrates: oligosaccharides. Thus, apart from fructooligosaccharides whose effects are most documented, fructans, galactooligosaccharides, xylooligosaccharides, soybean oligosaccharides, gentiooligosaccharides or also isomaltooligosaccharides, may be mentioned.

Fructooligosaccharides (FOS) are obtained either by hydrolysis of inulin, or by enzymatic synthesis, by transfructosylation from saccharide precursors. They correspond to the general formula Glucosyl-(Fructosyl)ₙ-Fructose or (Fructosyl)ₘ-Fructose where n represents an integer from 1 to 8 and m represents an integer from 1 to 8. In most cases FOS preparations are not homogeneous. They comprise mixtures of chains of variable size. Moreover, in the case of the preparation of FOS by enzymatic synthesis, the polymers correspond to the formula Glucosyl-(Fructosyl)ₙ-Fructose (1≦n≦8), while the FOS prepared by hydrolysis correspond to the two formulae Glucosyl-(Fructosyl)ₙ-Fructose and/or (Fructosyl)ₘ-Fructose (1≦n≦8 and 1≦m≦8). The FOS in particular comprise short-chain fructooligosaccharides, synthesized by transfructosylation, whose degree of polymerization is less than 6, and in particular short-chain FOS with 2, 3 or 4 fructose units such as 1-kestose, nystose and fructosyl-nystose.

Fructans are polymers in which the fructosyl-fructose type bonds predominate.

The galactooligosaccharides are formed by 2 to 6 hexose units, they mainly comprise galactose as a base unit. They are synthesized by the action of β-galactosidase on lactose.

Xylooligosaccharides originate from the hydrolysis of xylan, they are constituted by xylose.

Soybean oligosaccharides are extracted from soybean, these are mainly oligosaccharide mixtures comprising from 1 to 4 osidic units, the main constituents being raffinose and stachyose.

Gentiooligosaccharides are polymers originating from the digestion of starch, in which most of the bond has the β-glucopyranosyl-(1→6)-glucopyranose form.

Isomaltosaccharides are also glucose polymers which originate from the hydrolysis of starch, these are mixtures of isomaltose, panose, isomaltotriose and other branched polymers containing 4 or 5 glucose units.

The inventors have shown that the addition of prebiotics to the food intake, advantageously the addition of FOS, allowed a reduction in oxidative stress due in particular to a diet rich in sugars, and in particular fructose.

The purpose of the invention is to provide new means for preventing or treating oxidative stress.

A subject of the invention is the use of prebiotics for the preparation of food preparations, functional foods, or pharmaceutical compositions intended to prevent or treat oxidative stress.

A subject of the invention is more particularly the above-mentioned use of at least one oligosaccharide chosen from:
fructanes
fructooligosaccharides (FOS)
galactooligosaccharides
xylooligosaccharides
soybean oligosaccharides
gentiooligosaccharides
isomaltooligosaccharides

as defined above.

The invention more particularly relates to the above-mentioned use of fructooligosaccharides (FOS) of general formula Glucosyl-(Fructosyl)ₙ-Fructose or (Fructosyl)ₘ-Fructose where n represents an integer from 1 to 8, in particular from 1 to 5, and m represents an integer from 1 to 8, in particular from 1 to 5, such as short-chain FOS, 1-kestose, nystose or fructosyl-nystose.

A subject of the invention is also the use of prebiotics in the context of the prevention or treatment of oxidative stress linked to the consumption of sugars.

The invention more particularly relates to the use of prebiotics in the context of the prevention or treatment of oxidative stress linked to the consumption of fructose.

The invention in particular relates to the use of prebiotics in the context of the prevention or treatment of oxidative stress due to a consumption of fructose in food greater than approximately 50 g/day on average.

The invention also relates to the use of prebiotics, where said prebiotics are administered at a daily dose of approximately 1 g to approximately 20 g, in particular approximately 2 g to approximately 17 g, in particular approximately 5 g to approximately 15 g.

A subject of the invention is also the use of prebiotics as compounds with an anti-radical effect in the context of the prevention or treatment of oxidative stress.

A subject of the invention is also the use of prebiotics as compounds with an anti-aging effect linked to an effect which protects the cells of the organism against the action of free radicals.

The invention also relates to any food preparation comprising simple carbohydrates in combination with prebiotics.

The invention more particularly relates to a food preparation comprising:
at least one simple carbohydrate such as fructose or saccharose,
in combination with one or more oligosaccharides chosen from:
   fructanes
   fructooligosaccharides (FOS)
   galactooligosaccharides
   xylooligosaccharides
   soybean oligosaccharides
   gentiooligosaccharides
   isomaltooligosaccharides
as defined above.

Advantageously the food preparation of the invention is such that the proportion of prebiotics represents at least 5% by weight of the quantity of simple carbohydrates present in said preparation.

The invention in particular relates to a food preparation in which the proportion by weight of fructooligosaccharides (FOS) relative to the quantity of fructose present in said preparation varies between 10% and 100% and is in particular approximately 15% to approximately 35% and is in particular approximately 20%.

The invention in particular relates to a food preparation comprising a mixture of fructooligosaccharides (FOS), as defined above, comprising 64% Glucosyl-(Fructosyl)ₙ-Fructose and 36% (Fructosyl)ₘ-Fructose with average degrees of polymerization of 4.8.

The invention more particularly relates to a food preparation comprising a mixture of fructooligosaccharides (FOS), as defined above, comprising 64% Glucosyl-(Fructosyl)ₙ-Fructose and 36% (Fructosyl)ₘ-Fructose, with average degrees of polymerization of 4.8, the proportion by weight of said FOS present in said preparation varying between 10% and 100%, and in particular being approximately 15% to approximately 35%, preferably approximately 20%, relative to the quantity of fructose present in said preparation.

According to a preferred embodiment, the FOS mixture used corresponds to the Raftilose® P₉₅ preparation from ORAFTI, Thienen, Belgium.

A subject of the invention is also a food product containing the food preparation defined above, said food product being chosen from a group comprising pastries, confectionary, desserts, drinks, cereal bars, chocolate bars, sweet bars, breakfast cereals, dairy products and food supplements.

### DESCRIPTION OF THE INVENTION

The inventors have shown, in an animal model, that the addition of fructooligosaccharides (FOS) to the food intake allows a reduction in oxidative stress due to a diet enriched in fructose.

40 weaned male rats of the Wistar-Han type (IFFA-CREDO; L'Arbresle, France) 6 week old and weighing approximately 150 g were used. The rats were placed in cages with a wire mesh back in a temperature-controlled room (22° C.) with day/night cycles of 12 hours. The animals were treated according to the recommendations of the INRA Ethics Committee, decree No. 87-848.

First of all the rats were fed following a semi-purified starch-based diet for 7 days: They were then randomly divided into 4 groups of 10 rats: one starch group (A), one fructose group (F), one starch+FOS group (A/FOS) and one fructose+FOS group (F/FOS). They then followed their appropriate diet for 4 weeks.

The food and distilled water were provided ad libitum. The composition of the food rations was as follows (in g/kg):

| | Group | Group | Group | Group |
|---|---|---|---|---|
| | A | F | A/FOS | F/FOS |
| Starch | 650 | — | 550 | — |
| Fructose | — | 650 | — | 550 |
| FOS (Raftilose ® P₉₅) | — | — | 100 | 100 |
| Casein | 200 | 200 | 200 | 200 |
| Corn oil | 50 | 50 | 50 | 50 |
| Alphacel | 50 | 50 | 50 | 50 |
| Methionine D, L | 3 | 3 | 3 | 3 |
| Choline bitartrate | 2 | 2 | 2 | 2 |
| Mineral mixture (AIN-76) | 35 | 35 | 35 | 35 |
| Vitamin mixture (AIN-76A) | 10 | 10 | 10 | 10 |

The AIN-76 and AIN-76A mixtures were provided by ICN Biomedicals, Orsay, France.

The FOS (Raftilose® P₉₅) were obtained from ORAFTI, Thienen, Belgium. They were introduced into the food gradually in order to avoid diarrhoea which could occur in response to too rapid an administration of large quantities of this compound. Raftilose® P₉₅ is a mixture of Glucosyl-(Fructosyl)ₙ-Fructose (64%) and of (Fructosyl)ₘ-Fructose (36%) with average degrees of polymerization of 4.8.

4 days before sacrifice, the animals were housed individually in stainless steel cages with ad libitum access to the water and food. Urine-samples were recovered 24 hours before sacrifice in 50 ml graduated tubes, the volumes were precisely measured, the samples were then centrifuged and kept at −80° C. until analysis. At the time of sacrifice the rats were weighed, then anesthetized using sodium pentobarbital (intra-peritoneal injection at 40 mg/kg) and killed. The blood was taken from the abdominal aorta and placed in heparinized tubes. The plasma obtained after centrifugation at low speed (2000 g, 15 min) was kept at −80° C. for the biochemical analyses. The heart was rapidly removed then washed in an ice-cooled saline Solution (NaCl 9 g/l), placed in liquid nitrogen and kept at −80° C.

Two types of measurements well known to a person skilled in the art were then taken in order to determine the intensity of oxidative stress of the animals as a function of their diet: a measurement of the substances reactive to thiobarbituric acid (TBARS) and a measurement of the ratio of the plasma concentrations of vitamin E and triglycerides.

A statistical analysis of the results was carried out using the Statview program (Abacus Concepts Inc., Berkeley, Calif.). The data were expressed as the average of the results obtained for the 10 animals of each food group±standard deviation. The analysis of the variance (ANOVA; P<0.05) was used in order to determine the main effects (sugar and FOS) and their interactions. The differences were considered to be significant when p<0.05.

These results indicate that the animals following the fructose diet are subjected to a significantly greater oxidative stress than that of the control animals (subjected to the starch diet) and that the addition of FOS allows significant reduction in the oxidative stress linked to the consumption of fructose.

### EXAMPLE 1

Measurement of the Substances Reactive to Thiobarbituric Acid (TBARS)

Measurement of TBARS allows evaluation of the level of lipid peroxidation of a sample subjected to an oxidative stress. The greater the value of TBARS the higher the level of oxidative stress.

The levels of plasma TBARS were determined by spectrofluorometry on an LS 5 device (Perkin Elmer, Norwalk, Conn., USA). A method adapted from Okhawa et al. (1979) was used as previously described (Rayssiguier et al., 1993). The level of the urinary TBARS was measured as described in Lee et al. (1992) and calculated on the basis of a urinary volume of 24 hours. Finally, the measurement of the heart TBARS was based on Ohkawa et al. (1979), they allow the evaluation of the susceptibility of the heart lipids to peroxidation. The heart tissues were homogenized on ice at a ratio of 1 g of fresh tissue to 9 ml of KCl 150 mmol/l using a Polytron homogenizer, these homogenates were then subjected to a lipid peroxidation induced by a FeSO₄ (2 μmol/l)-ascorbate (50 μmol/l) mixture for 30 minutes in a bath at 37° C. in the absence of oxygen, a 1,1,3,3-tetraethoxypropane control was used; the TBARS were then measured by spectrophotometry (Uvikon 941 plus series, Kontron Instruments, St Quentin en Yvelines, France).

The results obtained are shown in the following table:

| | Diet | | | | Anova^{a} | | |
|---|---|---|---|---|---|---|---|
| | Starch | Fructose | Starch + FOS | Fructose + FOS | Sugar | FOS | Sugar × FOS |
| Plasma TBARS | 1.94 ± 0.03 | 2.14 ± 0.07 | 1.84 ± 0.02 | 1.96 ± 0.04 | <0.01 | <0.01 | NS |
| nmol/ml | | | | | | | |
| Urinary TBARS | 11.99 ± 0.50 | 21.97 ± 1.58 | 13.40 ± 0.53 | 15.86 ± 1.19 | <0.001 | <0.05 | <0.001 |
| nmol/24 h | | | | | | | |
| Heart TBARS | 64.9 ± 4.1 | 98.8 ± 6.5 | 73.1 ± 3.5 | 83.5 ± 4.7 | <0.001 | NS | <0.05 |
| nmol/g of fresh weight | | | | | | | |

The results are the averages calculated for 10 animals ± standard deviation.
^{a}value of p for the ANOVA. The results of the ANOVA are significant for p < 0.05, NS. not significant

The results indicate that the plasma, urinary and heart TBARS are significantly higher for the Fructose group than for the Starch group. The consumption of fructose is therefore responsible for greater oxidative stress than that which is due to the consumption of starch.

Moreover, the TBARS of the Fructose+FOS group are significantly lower than those of the Fructose group and are not significantly different to those of the Starch+FOS group. The FOS therefore allow the oxidative stress which is due to the consumption of fructose to be limited.

### EXAMPLE 2

Measurement of the Plasma Ratio of Vitamin E and Triglycerides

The ratio vitamin E/plasma triglycerides reflects the oxidative stress to which an organism has been subjected. The smaller the value of this ratio the greater the level of oxidative stress.

Measurement of the plasma triglyceride concentrations was carried out using enzymatic methods according to the recommendations of the supplier (Biotrol, Paris, France). A polyvalent control serum (Biotrol-33-plus) was treated at the same time as the samples in order to check the precision of the results of the plasma analysis.

The plasma concentrations of vitamin E were determined by reversed-phase high performance liquid chromatography on a Kontron series 400 device (Kontron St Quentin en Yvelines, France) using a hexane extract. α-tocopherol acetate (Sigma) was added to the samples as internal control. The samples were extracted twice with hexane after precipitation of the proteins with ethanol. The extract was dried under nitrogen, dissolved in an ethanol-methylene chloride mixture (65:35, v/v) and injected onto a C₁₈ column (Nucleosil; 250 mm long, i.d. 46 mm., 5 μm particles). Pure methanol allowed elution of the α-tocopherol in 5 minutes and the tocopherol acetate in 6.3 minutes at a flow rate of 2 ml/min. The compounds were detected by UV (292 nm) then quantified with internal and external calibrations using control solutions.

The obtained results are shown in the following table:

| | Diet | | | | Anova^{a} | | |
|---|---|---|---|---|---|---|---|
| | Starch | Fructose | Starch + FOS | Fructose + FOS | Sugar | FOS | Sugar × FOS |
| Triglycerides (TG) | 1.76 ± 0.21 | 3.73 ± 0.45 | 1.47 ± 0.11 | 2.49 ± 0.26 | <0.001 | <0.05 | NS |
| nmol/ml | | | | | | | |
| Vitamin E | 9.01 ± 0.54 | 9.74 ± 0.92 | 7.21 ± 0.35 | 8.74 ± 0.62 | NS | <0.05 | NS |
| μg/ml | | | | | | | |
| Vitamin E/TG | 5.98 ± 0.93 | 2.68 ± 0.12 | 5.03 ± 0.29 | 3.95 ± 0.61 | <0.001 | NS | NS |
| μg/mol TG | | | | | | | |

The results are the averages calculated for 10 animals ± standard deviation.
^{a}value of p for the ANOVA. The results of the ANOVA are significant for p < 0.05, NS. not significant

In contrast to the starch diet, the diet rich in fructose lowers the ratio Vitamin E/TG, which proves the existence of oxidative stress.

The supplementation with FOS prevents the lowering of this ratio, in other words reduces the oxidative stress which results from consumption of a diet rich in fructose.

### REFERENCES

Busserolles J., Rock E., Gueux E., Mazur A., Grolier P. and Rayssiguier Y., 2002a. Short term consumption of a high sucrose diet has a pro-oxydant effect in rats. Brit. J. Nutr., 87(4): 337-342.

Busserolles J., Gueux E., Rock E., Mazur A. and Rayssiguier Y., 2002b. Substituting honey for refined carbohydrates protects against the pro-oxydant effect of a high fructose diet. J. Nutr., 132(11): 3379-82.

Lee H. S., Shoeman D. W. and Csallany A. S., 1992. Urinary response to in vivo lipid peroxydation induced by vitamin E deficiency. Lipids, 27: 124-128.

Okhawa H., Ohishi N. and Yagi K., 1979. Assay for lipid peroxides in animal tissues by thiobarbituric acid reaction. Anal. Biochem. 95: 351-358.

Rayssiguier Y., Gueux E., Bussiere L., and Mazur A., 1993. Copper deficiency increases the suceptibility of lipoproteins and tissues to peroxidation in rats. J. Nutr. 123: 1343-1348.

1. A method of treatment for preventing or treating oxidative stress, comprising the use of prebiotics for the preparation of food preparations, functional foods, or pharmaceutical compositions.

2. The method of claim 1, comprising the use of at least one oligosaccharide chosen from:
fructans
fructooligosaceharides (FOS)
galactooligosaccharides
xylooligosaccharides
soybean oligosaccharides
gentiooligosaccharides
isomaltooligosaccharides

3. The method of claim 1, comprising the use of fructooligosaccharides (FOS) of general formula Glucosyl-(Fructosyl)ₙ-Fructose or (Fructosyl)ₘ-Fructose where n represents an integer from 1 to 8, in particular from 1 to 5, and m represents an integer from 1 to 8, in particular from 1 to 5, such as the short-chain FOS, 1 -kestose, nystose or fructosylnystose.

4. The method of claim 1, in the context of the prevention or treatment of oxidative stress linked to the consumption of sugars.

5. The method of claim 1, in the context of the prevention or treatment of oxidative stress linked to the consumption of fructose.

6. The method of claim 1, in the context of the prevention or treatment of oxidative stress which is due to a consumption of fructose in food greater than approximately 50 g/day on average.

7. The method of claim 1, in which said prebiotics are administered at a daily dose of approximately 1 g to approximately 20 g, in particular approximately 2 g to approximately 17 g, in particular approximately 5 g to approximately 15 g.

8. The method of claim 1, as compounds with an anti-radical effect in the context of the prevention or treatment of oxidative stress.

9. The method of claim 1, as compounds with an anti-ageing effect linked to an effect which protects the cells of the organism against the action of free radicals.

10. A food preparation comprising a mixture of fructooligosaccharides (FOS), as defined in claim 3, comprising 64% Glucosyl-(Fructosyl)ₙ-Fructose and 36% (Fructosyl)ₘ-Fructose, with average degrees of polymerization of 4.8, the proportion by weight of said FOS present in said preparation varying between 10% and 100%, and in particular being approximately 15% to approximately 35%, preferably approximately 20%, relative to the quantity of fructose present in said preparation.
